(19) United States
(12) Patent Application Publication
(10) Pub. No.: US 2016/0008262 A1
(43) Pub. Date: Jan. 14, 2016

(54) NATURAL BUTTERS RECONSTITUTED BY TRANSESTERIFICATION WITH GLYCERIN AND ITS USE IN COSMETICS APPLICATIONS

(71) Applicants: Arthur J. Hein, Morristown, NJ (US); Calvin M. Wicker,, JR., Spartanburg, SC (US); Charles Francis Palmer, JR., Greer, SC (US)

(72) Inventors: Arthur J. Hein, Morristown, NJ (US); Calvin M. Wicker,, JR., Spartanburg, SC (US); Charles Francis Palmer, JR., Greer, SC (US)

(21) Appl. No.: 14/857,708

(22) Filed: Sep. 17, 2015

Related U.S. Application Data
(63) Continuation of application No. 12/453,521, filed on May 14, 2009.

(51) Int. Cl. A61K 8/92; A61Q 5/12; A61K 8/06; A61Q 19/10; C07C 67/08; C07C 69/003; A61Q 5/02

(52) U.S. Cl. CPC A61K 8/922; C07C 69/003; A61Q 5/12; A61Q 5/02; A61Q 19/10; C07C 67/08; A61K 8/06

(57) ABSTRACT

The invention provides a composition comprising reaction products from a reaction of a natural butter or natural oil such as shea butter with glycerin in the presence of a basic catalyst and wherein the reaction products retain the unsaponifiable portion of said natural butter or natural oil. The resulting reaction products are self-emulsfiable and are particularly useful in personal care, cosmetic, pharmaceutical, paper and textuile applications.

This application is a continuation of pending application U.S. Ser. No. 12/453,521 entitled “Preparation Of A Shea Butter Reconstituted By Transesterification With Glycerin And Its Use In Cosmetics Applications” filed May 14, 2009; which is in its entirety herein incorporated by reference. This application also claims the priority benefit under 35 U.S.C. section 119 of U.S. Provisional Patent Application No. 61/071,699 entitled “Preparation Of A Shea Butter Reconstituted By Transesterification With Glycerin And Its Use In Cosmetics And Personal Care Applications” filed May 14, 2008, which is in its entirety herein incorporated by reference.

### FIELD OF INVENTION

The present invention concerns a self-emulsifying essentially hydrophobic formulation, namely, a formulation which upon mixture with water, spontaneously disintegrates to form an oil-in-water emulsion. This invention also generally relates to a process for preparing emulsions and cosmetics comprising such emulsions. This invention relates generally to the cosmetics field and, more particularly, to new self-emulsifying preparations which, even when used in very small quantities, lead to emulsions characterized by high storage and viscosity stability.

The invention further relates to a method for introducing high levels of functionality in exotic oils to aqueous based natural cosmetic formulations. The present invention relates to self-emulsifiable compositions. This invention also relates to the technical sector of cosmetic, dermatological, pharmaceutical, veterinary, paper, textiles and detergents. The present invention is also directed to unique green emulsions wherein the emulsifier is derived from natural butters and natural oils.

The invention also relates to improved cosmetic emulsions, especially to emulsions useful for moisturizing and conditioning the skin. This invention more particularly relates to cosmetic compositions using emulsifiers derived from natural butters and natural oils, especially to cosmetically acceptable skin lotions or creams having an emulsified base.

The present invention further relates to cosmetic and dermatological tissues which are moistened with highly liquid cosmetic and dermatological impregnation solutions—in particular with highly liquid cosmetic and dermatological water-in-oil emulsions (W/O emulsions) which are long-term stable.

Our invention relates to technology of transesterification of commercial oils commonly done with oils such as palm and coconut oils to work with exotic materials such as natural butters and natural oils and therefore create exotic glycerides, heretofore never done and providing benefits to cosmetic formulations. The prior art is silent on the reaction products and uses those products in cosmetic and personal care applications.

### BACKGROUND OF THE INVENTION AND DESCRIPTION OF THE PRIOR ART

Cosmetics are substances used to enhance, protect and beautify the appearance or odor of the human body. Cosmetics include shampoos, skin-care creams, lotions, powders, perfumes, lipsticks, fingernail and toe nail polish, eye and facial makeup, permanent waves, colored contact lenses, hair colors, hair sprays and gels, deodorants, baby products, bath oils, bubble baths, bath salts, butters and many other types of products. Their use is widespread, especially among women in Western countries. A subset of cosmetics is called “make-up,” which refers primarily to colored products intended to alter the user's appearance. Many manufacturers distinguish between decorative cosmetics and care cosmetics.

The manufacture of cosmetics is currently dominated by a small number of multinational corporations that originated in the early 20th century, but the distribution and sale of cosmetics is spread among a wide range of different businesses. The U.S. Food and Drug Administration (FDA) which regulates cosmetics in the United States defines cosmetics as: “intended to be applied to the human body for cleansing, beautifying, promoting attractiveness, or altering the appearance without affecting the body's structure or functions. This broad definition includes, as well, any material intended for use as a component of a cosmetic product. The FDA specifically excludes soap from this category.

The majority of cosmetic and pharmaceutical emulsions are of the oil-in-water type, i.e., the oil phase (“disperse phase”) is very finely distributed in the form of small droplets in the water phase (“coherent phase”). The viscosity of emulsions which consist only of water, oil and emulsifier, and whose content of disperse phase is below 60%, by weight, is equal to the viscosity of the coherent phase, and, in the case of oil-in-water emulsions, is thus equal to that of water. For reasons of feel on the skin, cosmetic emulsions on average comprise not more than 30% of oil phase, i.e., cosmetic emulsions are typically water-thin. Since, however, the consumer generally desires a lotion-like (high-viscosity) to cream-like (semisolid) consistency, and also the stability of emulsions increases with the viscosity of the coherent phase, the “thickening” of oil-in-water emulsions is essential. For this purpose there are two fundamentally different methods which can be combined with one another. The first method is based on the fact that certain oil-in-water emulsifiers are able, together with so-called “hydrophilic waxes”, to form liquid-crystalline (lamellar) structures in the coherent water phase. Moreover, this first method forms a three dimensional network which leads to a large increase in the viscosity of the emulsion; keeps the oil droplets separate from one another; and thus improves the stability of the emulsion. Examples of “hydrophilic waxes” are stearyl alcohol, stearic acid and glyceryl stearate.

The other method is based on the ability of so-called “hydrocolloids” to take up and bind many times their own weight of water and thus lead to thickening of water. Examples of such water-swellable organopolymers are crosslinked polyacrylates (“carbomers”) and polysaccharides, for example, xanthan gum. A disadvantage of these two thickening methods mentioned above is that the substances used therein can adversely affect the feel on the skin during or after application of the emulsions. Thus, for example, in the presence of relatively large amounts of hydrophilic waxes, the emulsions can only be spread with difficulty, and a dull, waxy feel on the skin often remains. On the other hand, the water-swellable organopolymers also display disadvantages in application properties. Thus, for example, in the case of carbomers, the so-called “quick-breaking effect” is observed. The “quick-breaking effect” is understood as the phenomenon where, in the case of contact of the emulsion with the electrolytes of the skin, the emulsion immediately breaks. This phenomenon is evident from an “aqueous sliding away” upon rubbing in and is often perceived as unpleasant.

For the preparation of oil-in-water emulsions, use is usually made of emulsifiers whose HLB value is between 8 and 18. The HLB value is a dimensionless parameter for characterizing surfactants and describes the ratio of the hydrophilic portion to the lipophilic portion in the molecule (HLB=hydrophilic-lipophilic balance). Thus, on the basis of numerous experiments by Griffin (J. Soc. Cosmet. Chem. 1949, 1, 311), it has been found that surfactants with an HLB value of 3 to 6 are suitable as water-in-oil emulsifiers, those with an HLB value of 6 to 8 are suitable as wetting agents, and surfactants with an HLB value of greater than 8 are suitable as oil-in-water emulsifiers. In the simplest case, the HLB value is calculated from the percentage proportion of the hydrophilic part of an emulsifier, for example, the polyethylene glycol part, by dividing this by 5. Thus, for example, the hydrophilic portion in the addition product of 20 mol of ethylene oxide (MW=880 g/mol) to stearic acid (284 g/mol) is 76%, corresponding to an HLB value of 15 (=76/5). This HLB concept has originally been limited to nonionogenic substances which contain no atoms other than carbon, hydrogen and oxygen. In addition, this HLB value definition does not apply exactly for substances whose hydrophilic part also contains propylene glycol units in addition to ethylene glycol units.

A disadvantage of emulsifiers with an HLB value of significantly greater than 8 is that such emulsifiers are less mild than emulsifiers with a lower HLB value. In addition, because of their higher hydrophilicity, emulsifiers having a HLB value of greater than 8 are more readily redispersible, i.e., they can be more readily washed off from the skin again with water, which, for example, in the case of sunscreen formulations that are supposed to be water-resistant, is undesired. Conversely, emulsifiers with an HLB value of around 8 and below form a hydrophobic film on the skin which protects the skin from excessive water loss and thus has a care effect. This is probably the main reason for the fact that water-in-oil emulsions, which require emulsifiers with an HLB value of less than 8, have a stronger care effect than oil-in-water emulsions that contain hydrophilic emulsifiers. However, oil-in-water emulsions are usually preferred by the consumer since oil-in-water emulsions can be spread more readily because of the aqueous external phase.

There has been a growing usage since the mid 1990's of natural butters, oils, fats, etc. in cosmetic applications. The most rapid growth has occurred in the last 5 years. The growth has occurred for 3 main reasons: Formulators wanting to make natural and exotic claims, the skin feel provided by the products and the innate functionality of the materials.

Typically these materials can be used in non aqueous based systems with relative ease. However in aqueous based systems (such as shampoos, conditioners and body washes) some type of emulsifier must be used to get the materials into formulation. Most all of these emulsifiers are non-natural materials that take away from natural claims and formulations, reduce the available amount of functionality and change skin feel. Accordingly, there is a long felt need to provide naturally derived emulsifiers which are sef-emulsifiable to addres the many deficicincies of the prior art.

### OBJECTS OF THE INVENTION

It is a primary object of the present invention to provide self-emulsifiable compositions derived from natural butters and natural oils.

It is another object of the present invention to provide reaction products derived from reacting natural butters and natural oils with glycerin.

It is also an object of the present invention to provide reaction products derived from reacting shea butters, shea oils and other natural oils with glycerin.

It is a further object of the present invention to provide self-emulsifiable compositions derived from shea butters and shea oils.

It is also an object of the present invention to provide personal care products containing reaction products derived from reacting natural butters and natural oils with glycerin.

Still, another object of the invention is to provide cosmetic products containing reaction products derived from reacting natural butters and natural oils with glycerin.

A further object of the invention is to provide personal care products containing the reaction products derived from reacting shea butters, shea oils and other natural oils with glycerin.

It is a specific object of the present invention to provide cosmetic products containing the reaction products derived from reacting shea butters, shea oils and other natural oils with glycerin.

Other objects and embodiments of the present invention will be further discussed below.

### SUMMARY OF THE INVENTION

This invention relates to a unique method of preparing an emulsion of shea butter in water so that it can be used in cosmetic applications. There are cosmetic treatments on the market that are based on Jojoba oil, beeswax, carnauba wax, candelila wax, and even castor oil that have been reconstituted by transesterification with various other triglycerides. The reasons for reconstituting these materials rather than use them as they are found in nature ranges from trying to achieve marketing advantages to trying to achieve product stabilities by adding unsaturation as an antioxidant. There are other treatments based on silicones as well. Typically, though, these compounds are rendered emulsifiable into water by the addition of emulsifiers and this is an additional step in the manufacture of these products. This invention is thought to be unique in that it has been found that reconstituting shea butter with glycerine alone so that the resulting reaction product is a mixture of mono- and di-esters and unreacted materials yields a composition that self-emulsifies into water to form a stable mixture that has a creamy appearance and sufficient viscosity so as not to separate, even on hot or cold storage.

The invention provides a composition comprising reaction products from a reaction of a natural butter or natural oil with glycerin in the presence of a basic catalyst and wherein the reaction products retain the unsaponifiable portion of said natural butter or natural oil.

The invention also provides a personal care product comprising appropriate ingredients in a liquid carrier, said personal care product including an effective emulsifying amount of the reaction products from the reaction of a natural butter or natural oil with glycerin in the presence of a basic catalyst and wherein the reaction products retain the unsaponifiable portion of said natural butter or natural oil.

The present invention also provides a cosmetic product comprising cosmetically acceptable ingredients in a liquid carrier, said product including an effective emulsifying amount of the reaction products from the reaction of a natural butter or natural oil with glycerin in the presence of a basic catalyst and wherein the reaction products retain the unsaponifiable portion of said natural butter or natural oil.

The instant invention also provides a method for making a natural butter or natural oil extract self-emulsifiable into water while retaining the unsaponifiable portion unchanged by reacting the natural butter or natural oil extract with glycerin to convert the triglycerides present to a mixture of mono- and diglycerides.

The invention further provides a self-emulsifiable composition comprising reaction products from a reaction of a natural butter or natural oil with glycerin in the presence of a basic catalyst and wherein the reaction products retain the unsaponifiable portion of said natural butter or natural oil.

The invention additionally provides a cosmetic or dermatological tissue comprising a water-insoluble nonwoven which is at least one of impregnated and moistened with a cosmetic or dermatological W/O emulsion, wherein the emulsion comprises (a) a water phase, (b) at least one oil phase which comprises one or more oils, one or more lipids and combinations thereof, and (c) an emulsifier system comprising the reaction product from a reaction of a natural butter or natural oil with glycerin in the presence of a basic catalyst and wherein the reaction products retain the unsaponifiable portion of said natural butter or natural oil.

The invention also relates to cosmetic or dermatological W/O emulsion for impregnating or moistening tissues, paper and textiles, wherein the emulsion comprises (a) a water phase, (b) at least one oil phase which comprises one or more oils, one or more lipids and combinations thereof, and (c) an emulsifier system comprising the reaction product from a reaction of a natural butter or natural oil with glycerin in the presence of a basic catalyst and wherein the reaction products retain the unsaponifiable portion of said natural butter or natural oil.

### DETAILED DESCRIPTION OF THE INVENTION

The invention provides a composition comprising reaction products from a reaction of a natural butter or natural oil with glycerin in the presence of a basic catalyst and wherein the reaction products retain the unsaponifiable portion of said natural butter or natural oil. The resulting products of the invention are exotic in nature and provide green solutions to the cosmetic and personal care industry.

The natural oil or natural butter selected from the group consisting of shea butter, shea stearin, shea olein, shea oil, mango butter, mango oil, mango olein, cupuacu butter, cocoa butter, illipe butter, acai oil, buriti oil, andiroba oil, almond oil, avocado oil, evening primrose oil, manila oil, argan oil, tea tree oil, jojoba oil, macadamia oil, chia oil, brazil nut oil, annato oil, copaiba oil, passionfruit oil, murumuru butter, ucuuba butter, tucuma butter, tucuma oil, pequi oil, sangue de dragão oil, and patua oil, croton oil, mulaterio oil, unha de gato oil, breu branco oil, croton oil, pracaxi oil, urucum oil, and other natural oil extracts containing triglycerides.

The basic catalyst for the process is selected from the group consisting of sodium hydroxide and potassium hydroxide. However, other basic catalyst may be used in the practice of the invention such as calcium hydroxide, sodium methoxide or potassium methoxide.

In a typical preparation, the natural butter and natural oil having melting points of 35° C. and 15° C. were reacted with anhydrous glycerin at the ratio of 90.55% shea butter to 8.96% glycerin with 0.5% KOH flake as a catalyst. The reaction is conducted in a suitable reactor either at ambient temperature or heated to a temperature sufficient to promote transesterification and preferably in the range 130°-200° C.

In one embodiment of the invention, shea butters with melting points of 35° C. and 15° C. were reacted using the above stated temperature range with anhydrous glycerin at the ratio of 90.55% shea butter to 8.96% glycerin with 0.5% KOH flake as a catalyst to produce a shea butter product hereinafter designated as Jarplex SB-WD is composed of the glycerides of vegetable fat extracted from the fruit of the Shea tree (Butyrospermum parkii). Jarplex SB-WD contains a relatively high amount of unsaponifiable matter and is processed to have extremely low odor and light color. It contributes high levels of shea functionality to a wide variety of formulations. Jarplex SB-WD is completely water dispersible in aqueous-based formulations, as well as compatible with surfactant based systems. It is also self-emulsifying.

The resulting Jarplex SB-WD has a maximum Color (Gardner value) not exceeding 10, a maximum acid value not exceeding 3 (mg KOH/g), a saponification value in the range of 150-165 (meq/kg), a pH in the range of 6.0-8.0 and a moisture content not exceeding 1.0%. The resulting products Jarplex SB-WD have melting points in the range of 40°-44° C., an unsaponifiable content of 6-8% and a peroxide value of <5.0 (meq/kg) and HLB values of 4.5-5.5.

Jarplex SB-WD is well suited for diverse cosmetic and dermatological applications, ensuring more reliable visual and skin feel aesthetics over a wide range of aqueous and non-aqueous conditions. Due to its superior water dispersion properties, Jarplex SB-WD is an effective emulsifier for all kinds of natural vegetable oils. It is especially well suited for use in formulations with high concentrations of natural exotic materials. Jarplex SB-WD produces unique feeling, “rapid absorbing” creams and lotions with a soothing non-greasy feel. Because it is made from natural vegetable lipid materials, Jarplex SB-WD is non-toxic, non-irritating and readily biodegradable. It can be employed in skin care formulations including body washes, shampoos, conditioners, creams, lotions, sunscreen products, massage creams, and ointments. Typical application levels are from 1-20%. Jarplex SB-WD alone at 25% in water produces a luxurious lotion.

In another embodiment of the invention, cupuacu butters were reacted with anhydrous glycerin at the ratio of 90.00% cupuacu butter to 10.00% glycerin with 0.5% KOH flake as a catalyst to produce a shea butter product hereinafter designated as Jarplex CU-WD is composed of the glycerides of vegetable fat extracted from the fruit of the Shea tree (Butyrospermum parkii). Jarplex CU-WD contains a relatively high amount of unsaponifiable matter and is processed to have extremely low odor and light color. It contributes high levels of shea functionality to a wide variety of formulations. Jarplex CU-WD is completely water dispersible in aqueous-based formulations, as well as compatible with surfactant based systems. It is also self-emulsifying.

The resulting Jarplex CU-WD has a maximum Color (Gardner value) not exceeding 20, a maximum acid value not exceeding 3 (mg KOH/g), a saponification value in the range of 160-180 (meq/kg), a pH in the range of 6.5-8.5 and a moisture content not exceeding 1.0%. The resulting products Jarplex CU-WD have melting points in the range of 40°-44° C., an unsaponifiable content of 6-8% and a peroxide value of <5.0 (meq/kg) and HLB values of 4.5-5.5.

Jarplex CU-WD is well suited for diverse cosmetic and dermatological applications, ensuring more reliable visual and skin feel aesthetics over a wide range of aqueous and non-aqueous conditions. Due to its superior water dispersion properties, Jarplex CU-WD is an effective emulsifier for all kinds of natural vegetable oils. It is especially well suited for use in formulations with high concentrations of natural exotic materials. Jarplex CU-WD produces unique feeling, “rapid absorbing” creams and lotions with a soothing non-greasy feel. Because it is made from natural vegetable lipid materials, Jarplex CU-WD is non-toxic, non-irritating and readily biodegradable. It can be employed in skin care formulations including body washes, shampoos, conditioners, creams, lotions, sunscreen products, massage creams, and ointments. Typical application levels are from 1-20%. Jarplex CU-WD alone at 25% in water produces a luxurious lotion.

The resultant transester mixtures of both Jarplex SB-WD and Jarplex CU-WD of the invention were blended at 5% and 10% levels in tap water and both yielded stable emulsions or creams.

The products of the invention are useful in personal care applications, cosmetic applications, paper and textile applications as well as pharmaceutical applications including dermatological applications.

These products are easy to use, since they need limited amount of heating for use. Approximately 45 C, compared to 70 C for other materials. The application of these exotic glycerides allow incorporation of exotic materials into aqueous based cosmetic formulations with ease. The exotic glycerides are all natural and green and allow formulators to make these natural claims. The unsaponifiable fractions that give the exotic materials their functionality are completely carried over in this glyceride technology.

These exotic glycerides are self emulsifying and carry the innate functionality totally to formulations. These products also emulsify other exotic oils and fats which will even further boost the functionality of the end formulations. High amounts (>15%) in water alone, form luxurious stable natural lotions.

Applicant's have also found that varying the glyceride mole ratio will also vary the water solubility to have addition flexibility in formulation.

Other embodiments of the invention include 1%, 2%, 5%, 10%, and 15% by weight of shea glycerides in water which are water dispersible and stable. Shea butter itself is non-dispersible in water. Different cosmetics formulas using shea glycerides and Cupuaçu glycerides such as Shampoo, Body Wash, Conditioner, Body Butter, Lotion are exemplified in the Examples further below.

Active shea functionality in shea glycerides vs. shea ethoxylate, vs. shea betaine vs. some others are also of ineterest.

Other exotic oils which can be modified by this invention includes shea olein, mango butter, mango oil, illipe butter, acai oil, burriti oil, andiroba oil, brazil nut oil, annato oil, copaiba oil, passionfruit oil, murumuru butter, ucuuba butter, tucuma butter, pequi oil, sangue de dragão oil, patua oil and others.

The glyceride portion of the resulting products of the invention can be derived from glycerine as well as polyglycerol and other polyhydric alcohols.

The present invention also relates to the use of the self-emulsifying preparations of the invention for the production of cosmetic, personal care and/or pharmaceutical compositions in which they may be present in quantities of 0.1 to 25% by weight and preferably 0.5 to 20% by weight, based on the preparation.

The cosmetic and/or pharmaceutical compositions prepared using the self-emulsifying preparations may contain other typical auxiliaries and additives such as, for example, mild surfactants, oil components, emulsifiers, pearlizing waxes, consistency factors, thickeners, superfatting agents, stabilizers, polymers, silicone compounds, fats, waxes, lecithins, phospholipids, UV protection factors, moisturizing components, biogenic agents, antioxidants, deodorizers, antiperspirants, anti-dandruff agents, film formers, swelling agents, insect repellents, self-tanning agents, tyrosine inhibitors (depigmenting agents), hydrotropes, solubilizers, preservatives, perfume oils, dyes and the like.

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, .alpha.-methyl ester sulfonates, sulfofatty acids, alkyl sulfates, alkyl ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, .alpha.-olefin sulfonates, ether carboxylic acids, alkyl oligoglucosides, fatty acid glucamides, alkylamidobetaines, amphoacetals and/or protein fatty acid condensates, preferably based on wheat proteins.

In making topicasl compsotions for the skin, the emulsions of the invention also include oils chosen from polar oils, for example from lecithins and fatty acid triglycerides, especially the triglycerol esters of saturated and/or unsaturated, branched and/or unbranched alkanecarboxylic acids of a chain length of 8 to 24, in particular 12 to 18, carbon atoms. The fatty acid triglycerides can, for example, be chosen advantageously from synthetic, semisynthetic and natural oils, such as, for example, coconut glyceride, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil and the like.

The emulsions of the invention also include preservatives. Within the meaning of the present invention are, for example, formaldehyde-cleaving agents (such as, for example, DMDM hydantoin, iodopropyl butylcarbamate, parabens, phenoxy-ethanol, ethanol, benzoic acid and suchlike. Customarily, according to the invention the preservative system advantageously also contains preservation aids, such as, for example, ethylhexyloxyglycerol, Glycine, etc.

In addition, humectants or “moisturizers” can be present. Moisturizers are designated as substances or substance mixtures which impart to cosmetic or dermatological preparations the property, after the application to or dispersion on the skin surface, of reducing the release of moisture from the horny layer (also called trans-epidermal water loss (TEWL)) and/or of positively influencing the hydration of the horny layer.

Advantageous moisturizers within the meaning of the present invention are, for example, glycerol, lactic acid, pyrrolidonecarboxylic acid and urea. Furthermore, it is particularly advantageous to use polymeric moisturizers from the group consisting of the polysaccharides which are water-soluble and/or swellable in water and/or gellable with the aid of water. Those particularly advantageous are, for example, hyaluronic acid, chitosan and/or a fucose-rich polysaccharides.

The cosmetic or dermatological preparations according to the invention can furthermore advantageously, even though not compulsorily, contain fillers which, for example, further improve the sensory and cosmetic properties of the formulations and, for example, produce or increase a velvety or silky skin sensation. Advantageous fillers within the meaning of the present invention are starch and starch derivatives (such as, for example, tapioca starch, distarch phosphate, aluminum or sodium starch octenylsuccinate and the like), pigments which have neither mainly UV filter nor coloring action (such as, for example, boron nitride etc.).

The mono-di-transesters of the invention also have a unique set of properties that would make it of significant interest to the textile, paper and tissue industries. It's inherent water dispersibility would provide significant advantages to the consumer versus traditional butters and oils.

The application or incorporation of hydrophobic materials into cellulosic webs (Paper, tissue, etc.) has traditionally been very problematic. For example since 1977 several methods have been developed to emulsify alkenyl succinic anhydride for use as a water-proofing agent in paper. Various methods of spraying or extruding oils and viscous materials onto a paper web have also been developed.

The primary issues are: (1) surfactants, which are typically used to incorporate hydrophobic materials into aqueous systems, cause foaming which adversely affects the efficiency of the papermaking process, (2) efficient retention and dispersion of hydrophobic materials in the paper web is difficult to achieve and (3) completely water soluble materials are difficult to remove with traditional water treatment processes.

The mono-di-transesters of the invention are potentially much easier to apply to paper webs addressing these issues because:

(1) they do not use surfactants and should cause less foaming in the papermaking process,

(2) aqueous dispersions of mono-di-transesters are made up of small particulates. These particulates, especially if sprayed onto the paper web versus addition to the papermaking stock system, would be physically entrapped in the paper web increasing it's retention in the paper above that which results from simple ionic attraction and flocculation,

(3) Since mono-di-transesters are not completely soluble, as are, for example, ethoxylated oils, separation and removal during normal water treatment processes should be readily achieved.

In one embodiment, a shea mono-di-transeters is mixed at 5% concentration (wt/wt) with hot water or dilute stock white water using standard mixing/agitation equipment. The resulting dispersion is sprayed onto a paper tissue web just prior to the wet line on a traditional fourdrinier paper machine. The resulting tissue would have the emollient characteristics of shea butter incorporated into the web and that would be transferred to the consumer upon use.

In another embodiment, shea mono-di-transeters is heated to melting and mixed at 5% concentration (wt/wt) with warm water by shaking in a small spray bottle. The resulting dispersion can be sprayed onto a textile substrate (i.e. wash cloth, inside surface of a robe). Due to it's particulate nature the shea butter will tend to remain on the surface of the substrate rather than penetrating. The emollient characteristics of the shea butter is transferred to the consumer upon use. Further, as the oil is water dispersible, removal of the material by standard home clothes washing methods is enhanced.

### EXAMPLES

The compositions and uses of the invention are illustrated by the following examples, which are merely indicative of the nature of the present invention, and should not be construed as limiting the scope of the invention, nor of the appended claims, in any manner.

### Example I

Shea butters with melting points of 35° C. and 15° C. were reacted with anhydrous glycerin at the ratio of 90.55% shea butter to 8.96% glycerin with 0.5% KOH flake as a catalyst. The resulting product has a maximum Color (Gardner value) not exceeding 10, a maximum acid value not exceeding 3 (mg KOH/g), a saponification value in the range of 150-165 (meq/kg), a pH in the range of 6.0-8.0 (1% IPA:H2O) and a moisture content not exceeding 1.0%. The resulting products have melting points in the range of 40°-44° C., an unsaponifiable content of 6-8% and a peroxide value of <5.0 (meq/kg) and HLB values of 4.5-5.5. This material hereinafter is designated the name of Jarplex SB-WD.

### Example II

Cupuaçu butters were reacted with anhydrous glycerin at the ratio of 90.00% Cupuaçu butter to 10.00% glycerin with 0.5% KOH flake as a catalyst. The resulting product has a maximum Color (Gardner value) not exceeding 20, a maximum acid value not exceeding 3 (mg KOH/g), a saponification value in the range of 160-180 (meq/kg), a pH in the range of 6.5-8.5 and a moisture content not exceeding 1.0%. The resulting products have melting points in the range of 40°-44° C., an unsaponifiable content of 6-8% and a peroxide value of <5.0 (meq/kg) and HLB values of 4.5-5.5. This material hereinafter is designated the name of Jarplex CU-WD.

### Example IIA

Mango butters were reacted with anhydrous glycerin at the ratio of 90.00% Cupuaçu butter to 10.00% glycerin with 0.5% KOH flake as a catalyst. The resulting product has a maximum Color (Gardner value) not exceeding 20, a maximum acid value not exceeding 3 (mg KOH/g), a saponification value in the range of 160-180 (meq/kg), a pH in the range of 6.5-8.5 and a moisture content not exceeding 1.0%. The resulting products have melting points in the range of 40°-44° C., an unsaponifiable content of 6-8% and a peroxide value of <5.0 (meq/kg) and HLB values of 4.5-5.5. This material hereinafter is designated the name of Jarplex CU-WD.

### Example III

### Hair Conditioner

| INGREDIENTS | INCI Name | Function | % |
|---|---|---|---|
| PART A | | | |
| R.O. Water | Water | Solvent | 41.50 |
| Jaguar C17 | Guar Hydroxypropyl- | Viscosity | 0.80 |
| | trimonium Chloride | Control | |
| Citric Acid (25% | Citric Acid | | 0.20 |
| aq soln) | | | |
| PART B | | | |
| Genamin KDMP | Behentrimonium Chloride | Conditioner | 1.00 |
| JARPLEX SB-WD | Butyrospermum Parkii | Conditioner | 5.00 |
| | (Shea Butter) Glycerides | | |
| JARPLEX SB35 | Butyrospermum Parkii | Conditioner | 1.00 |
| | (Shea Butter) | | |
| PART C | | | |
| Fragrance 115620 | Fragrance | Fragrance | 0.20 |
| Color | | Color | QS |
| Germaben II E | Propylene Glycol (and) | Preservative | 1.00 |
| | Diazolidinyl Urea (and) | | |
| | Methylparaben (and) | | |
| | Propylparaben | | |
| | | | 100 |

Procedure:

Add the Part A water to a suitable vessel. Begin mixing and sprinkle in the Jaguar. Mix until uniform and add the citric acid. Continue mixing and add the remainder of Part A. Continue mixing and heat to 75-80° C. Mix until uniform. Continue mixing and stop heating.

Continue mixing and add Part B in order. Mix until uniform avoiding aeration. Continue mixing and cool to 45° C. Continue mixing and add the Part C ingredients. Mix until uniform. The pH is ˜3.4 and the Viscosity ˜5,200 cps Sp #3 @ 30 rpm.

### Example IV

### Bath Gel

| INGREDIENTS | INCI Name | Function | % |
|---|---|---|---|
| PART A | | | |
| R.O. Water | Water | Solvent | 52.90 |
| Xanthan Gum | Xanthan Gum | Viscosity | 0.8 |
| | | Control | |
| Citric Acid (25% | Citric Acid | pH Control | 0.10 |
| aq soln) | | | |
| PART B | | | |
| Plantaren 2000N UP | Decyl Glucoside | Surfactant | 5.00 |
| | Hydroxysultaine | | |
| JARPLEX SB-WD | Butyrospermum Parkii | Conditioner | 3.00 |
| | (Shea Butter) Glycerides | | |
| Steol CS-230 | Sodium Laureth Sulfate | Surfactant | 20.00 |
| Crodasinic LS-30 | Sodium Lauroyl Sarcosinate | Surfactant | 10.00 |
| Crosultaine C-50 | Cocamidopropyl | Surfactant | 7.00 |
| | Hydroxysultaine | | |
| PART C | | | |
| Fragrance 115620 | Fragrance | Fragrance | 0.20 |
| Color | — | Color | QS |
| Germaben II E | Propylene Glycol (and) | Preservative | 1.00 |
| | Diazolidinyl Urea (and) | | |
| | Methylparaben (and) | | |
| | Propylparaben | | |
| | | | 100 |

Procedure:

Add the Part A water to a suitable vessel. Begin mixing and sprinkle in the Xanthan Gum. Mix until uniform and add the citric acid. Continue mixing and heat to 65-70° C. Mix until uniform. Continue mixing and stop heating. Continue mixing and add Part B in order. Mix until uniform avoiding aeration. Continue mixing and cool to 45° C. Continue mixing and add the Part C ingredients. Mix until uniform. pH as is ˜7.8 Viscosity ˜2,550 cps Sp #3 @ 12 rpm.

### Example V

### Conditioning Shampoo

| INGREDIENTS | INCI Name | Function | % |
|---|---|---|---|
| PART A | | | |
| R.O. Water | Water | Solvent | 41.50 |
| Xanthan Gum | Xanthan Gum | Viscosity | 0.70 |
| | | Control | |
| Citric Acid (25% | Citric Acid | pH Control | 0.10 |
| aq soln) | | | |
| PART B | | | |
| Crosultaine C-50 | Cocamidopropyl | Surfactant | 12.50 |
| | Hydroxysultaine | | |
| JARPLEX SB-WD | Butyrospermum Parkii | Conditioner | 4.00 |
| | (Shea Butter) Glycerides | | |
| Steol CA-30 | Sodium Laureth Sulfate | Surfactant | 30.00 |
| Stepanol CA | Sodium Lauryl Sulfate | Surfactant | 10.00 |
| PART C | | | |
| Fragrance 115620 | Fragrance | Fragrance | 0.20 |
| Color | — | Color | QS |
| Germaben II E | Propylene Glycol (and) | Preservative | 1.00 |
| | Diazolidinyl Urea (and) | | |
| | Methylparaben (and) | | |
| | Propylparaben | | |
| | | | 100 |

### Procedure:

Add the Part A water to a suitable vessel. Begin mixing and sprinkle in the Xanthan Gum. Mix until uniform and add the citric acid. Continue mixing and add the remainder of Part A. Continue mixing and heat to 65-70° C. Mix until uniform. Continue mixing and stop heating.

Continue mixing and add Part B in order. Mix until uniform avoiding aeration. Continue mixing and cool to 45° C. Continue mixing and add the Part C ingredients. Mix until uniform. pH as is 5.8 and Viscosity ˜4,000 cps Sp #3 @ 30 rpm.

### Example VI

### 25% Butter Cream

| INGREDIENTS | INCI Name | Function | % |
|---|---|---|---|
| PART A | | | |
| R.O. Water | Water | Solvent | 63.91 |
| Glycerin | Glycerin | Moisturiza- | 3.00 |
| | | tion | |
| Pemulen TR-2 | Acrylates/C10-30 Alkyl | Viscosity | 0.20 |
| | Acrylate Crosspolymer | | |
| NaOH (50% aq. | Sodium Hydroxide | pH Control | 0.04 |
| Soln) | | | |
| PART B | | | |
| JARPLEX SB35 | Butyrospermum Parkii | Emollient | 25.00 |
| | (Shea Butter) | | |
| JARPLEX SB-WD | Butyrospermum Parkii | Emollient | 5.00 |
| | (Shea Butter) Glycerides | | |
| DowCorning 200 | Dimethicone | Emollient | 1.00 |
| Fluid (1000 cps) | | | |
| Polyaldo 10-1-10 | Polyglyceryl-10 Oleate | Emulsifier | 0.50 |
| Mixed Tocopherols | Tocopherols | Antioxidant | 0.20 |
| 70 | | | |
| PART C | | | |
| Fragrance SZ18197 | Fragrance | Fragrance | 0.10 |
| Yland YIang | Fragrance | Fragrance | 0.05 |
| Color | — | Color | QS |
| Germaben II E | Propylene Glycol (and) | Preservative | 1.00 |
| | Diazolidinyl Urea (and) | | |
| | Methylparaben (and) | | |
| | Propylparaben | | |
| | | | 100 |

### Procedure:

Add the Part A water to a suitable vessel. Begin mixing and sprinkle in the Jaguar. Mix until uniform and add the citric acid. Continue mixing and add the remainder of Part A. Continue mixing and heat to 75-80° C. Mix until uniform. Continue mixing and stop heating. Continue mixing and add Part B in order. Mix until uniform avoiding aeration. Continue mixing and cool to 45° C. Continue mixing and add the Part C ingredients. Mix until uniform. pH as is ˜3.4 and Viscosity ˜5,200 cps Sp #3 @ 30 rpm.

### Example VII

### Moisturizing Lotion

| INGREDIENTS | INCI Name | Function | % |
|---|---|---|---|
| PART A | | | |
| R.O. Water | Water | Solvent | 81.85 |
| Glycerin | Glycerin | Moisturiza- | 2.00 |
| | | tion | |
| Ultrez 10 | Carbomer | Viscosity | 0.30 |
| Triethanolamine | Triethanolamine | pH Control | 0.50 |
| 99% | | | |
| PART B | | | |
| JARPLEX SB-WD | Butyrospermum Parkii | Emollient | 3.00 |
| | (Shea Butter) Glycerides | | |
| Safflower Oil | Carthamus Tinctorius | Emollient | 5.00 |
| | (Safflower) Seed oil | | |
| Apricot Kernel Oil | Prunus Armeniaca | Emollient | 2.00 |
| | (Apricot) Kernel oil | | |
| Olive Oil | Olea Europaea | Emollient | 2.00 |
| | (Olive) Fruit oil | | |
| Jojoba Oil | Simmondsia Chinensis | Emollient | 1.00 |
| | (Jojoba) seed oil | | |
| Stearic Acid | Stearic Acid | Emuslsifier | 1.00 |
| Mixed Tocopherols | Tocopherols | Antioxidant | 0.20 |
| 70 | | | |
| PART C | | | |
| Fragrance SZ18197 | Fragrance | Fragrance | 0.10 |
| Yland YIang | Fragrance | Fragrance | 0.05 |
| Color | — | Color | QS |
| Germaben II E | Propylene Glycol (and) | Preservative | 1.00 |
| | Diazolidinyl Urea (and) | | |
| | Methylparaben (and) | | |
| | Propylparaben | | |
| | | | 100 |

### Procedure:

Add the Part A water to a suitable vessel. Begin mixing and sprinkle in the Ultrez. Mix until uniform and add the TEA. Continue mixing and add the remainder of Part A. Continue mixing and heat to 65-70° C. Mix until uniform. Continue mixing and stop heating. Combine the Part B ingredients and heat with mixing to 65-70° C. Continue mixing Part A and add Part B. Mix for at least 15 minutes at temperature. Continue mixing and cool to 45° C. Continue mixing and add the Part C ingredients. Mix until uniform. pH as is ˜7.25 and Viscosity ˜180,000 cps Sp #3 @ 0.6 rpm.

### Example VIII

### Oil in Water Emulsions

### Preparation of Emulsions:

10% Oil-in-water emulsions were made from all four of the transesters shown below by the following procedure:

| | SB-35 | SB-15 | Mango | Cupuacu |
|---|---|---|---|---|
| | transester | transester | transester | transester |
| | Melting pt. | Melting pt. | Melting pt | Melting point |
| Raw material | 38 C. | 36 C. | 35 C. | 40 C. |
| Shea Butter | 90.55% | | | |
| 35 C. melt | | | | |
| Shea Butter | | 90.55% | | |
| 15 C. melt | | | | |
| Mango Oil | | | 90.55% | |
| Capuacu Butter | | | | 90.55% |
| KOH Flake | 00.50% | 00.50% | 00.50% | 00.50% |
| Glycerin | 08.96% | 08.96% | 08.96% | 8.96% |

1) Preheat DI water to 60 C and hold there with a thermocouple temperature controller
2) Premelt the transesters with 3 thirty-second microwave bursts followed by storeage at 70 C
3) Start overhead blade mixer in 180 grams of the preheated DI water, then add with continued mixing, 20 grams of molten transester.
4) Mix without additional heating, at moderate speeds for 1 hour.
5) Every 15 minutes, mix at high speeds with a drammel homogenizer for 30 seconds.
6) At the end of the hour, the mix was cooled to ambient temperature.
In all cases the resulting emulsions were white and creamy with a viscosity or 1000-1500 cps and did not separate in 72 hours. Also, all diluted further in water with ease.
This test was repeated with same stabilities and results for 1%, 2%, 5%, 10%, 15% and 25%.

### Example IX

### Unstable Oil in Water Emulsions

Oil-in-water emulsions were not stable which were made using oil phases composed of mixtures of shea butters and shea butters reacted with non-ionic hydrophilic groups. The oil-phase mixtures were prepared as follows: A shea butter of 15 C melting point was reacted in two ways with hydrophilic groups. First, the shea butter was tranesterified, one-mole-to-one-mole, with glycerin. Next, a mole of this transester was reacted with 50 moles of ethylene oxide.

The table below describes the mixtures generated.

| Raw materials | A | B | C | D |
|---|---|---|---|---|
| Shea Butter 35 C. Melting | 50% | | 50% | |
| Point (Solid at ambient) | By weight | | By weight | |
| Shea Butter 15 C. Melting | | 50% | | 50% |
| Point (Liquid at ambient) | | By weight | | By weight |
| POE (50) Shea Butter | 50% | 50% | | |
| transester | | | | |
| Shea butter/glycerine ester | | | 50% | 50% |
| Appearance of mix | Uniform | Uniform | Uniform | Uniform |
| | wax | wax | wax | wax |

The emulsions were prepared as follows and the descriptions are shown in the table immediately following:

1) Heat all the butter samples—reacted and unreacted—to 110 deg C
2) Heat a large stock of water to 65 deg C on a hot plate with a constant temperature probe.
3) Premix the reacted and unreacted shea butters
4) Add the Shea Butter mixes to the hot water with mixing
5) Allow the water emulsions to cool with mixing
6) Allow to stand 24 hours

| Emulsion Descriptions | | | | |
|---|---|---|---|---|
| after 24 hours | A | B | C | D |
| Appearance of 2.5% in water | Wax | Wax | Cream- | Creaming |
| | separation | separation | ing | |
| | at surface | at surface | | |
| Appearance of 10% in water | Wax | Wax | Cream- | Creaming |
| (Made by the procedure in | separation | separation | ing | |
| example I) | at surface | at surface | | |

### Example X

### Unstable Oil in Water Emulsions

Oil-in-water emulsions were not stable which were made using oil phases composed of mixtures of shea butters and traditional emulsifiers. The compositions of the various oil phases are tabulated below with the descriptions of the emulsions at the lower part of the table.

| | A | | | | | | | | | |
|---|---|---|---|---|---|---|---|---|---|---|
| | % by | | | | | | | | | |
| Raw materials | weight | B | C | D | E | F | G | H | I | J |
| Shea Butter SB 35 | 50 | 50 | 50 | 50 | X | X | X | X | 50 | X |
| Shea Butter SB 15 | X | X | X | X | 50 | 50 | 50 | 50 | X | 50 |
| POE(100)monostearate | X | X | X | 50 | X | X | X | 50 | X | X |
| Ethsorbox O-20 | 50 | X | X | X | 50 | X | X | X | 50 | X |
| Ethsorbox L-20 | X | 50 | X | X | X | 50 | X | X | X | X |
| Ethox GMO-15 | X | X | 50 | X | X | X | 50 | X | X | X |
| Ethox CO-200 | X | X | X | X | X | X | X | X | 50 | 50 |
| Appearance of mix | Soft | Hard | Paste | Wax | Soft | Liquid | Liquid | Wax | hard | Soft |
| | paste | paste | sepaReated | | paste | split | Split | | paste | paste |
| | split | | | | | | | | | solid |
| Appearance of | Hard | Hard | Cream | Powder | Cream | Surface | Cream | Cream/oil | Hard | Cream |
| 2.5% emulsion after | cream | wax | wax | surface | sep | particles | sep | sep. | wax | oil |
| 24 hours | sep | sep | sep | | | | | | sep | |

### Example XI

### Hair Conditioner

| INGREDIENTS | INCI Name | Function | % |
|---|---|---|---|
| PART A | | | |
| R.O. Water | Water | Solvent | 41.50 |
| Jaguar C17 | Guar Hydroxypropyl- | Viscosity | 0.80 |
| | trimonium Chloride | Control | |
| Citric Acid (25% | Citric Acid | | 0.20 |
| aq soln) | | | |
| PART B | | | |
| Genamin KDMP | Behentrimonium Chloride | Conditioner | 1.00 |
| JARPLEX CU-WD | Cupuaçu Butter Glycerides | Conditioner | 5.00 |
| JARPLEX SB35 | Butyrospermum Parkii | Conditioner | 1.00 |
| | (Shea Butter) | | |
| PART C | | | |
| Fragrance 115620 | Fragrance | Fragrance | 0.20 |
| Color | | Color | QS |
| Germaben II E | Propylene Glycol (and) | Preservative | 1.00 |
| | Diazolidinyl Urea (and) | | |
| | Methylparaben (and) | | |
| | Propylparaben | | |
| | | | 100 |

Procedure:

Add the Part A water to a suitable vessel. Begin mixing and sprinkle in the Jaguar. Mix until uniform and add the citric acid. Continue mixing and add the remainder of Part A. Continue mixing and heat to 75-80° C. Mix until uniform. Continue mixing and stop heating.

Continue mixing and add Part B in order. Mix until uniform avoiding aeration. Continue mixing and cool to 45° C. Continue mixing and add the Part C ingredients. Mix until uniform. The pH as is ˜3.4 and Viscosity ˜5,200 cps Sp #3 @ 30 rpm.

### Example XII

### Bath Gel

| INGREDIENTS | INCI Name | Function | % |
|---|---|---|---|
| PART A | | | |
| R.O. Water | Water | Solvent | 52.90 |
| Xanthan Gum | Xanthan Gum | Viscosity | 0.8 |
| | | Control | |
| Citric Acid (25% | Citric Acid | pH Control | 0.10 |
| aq soln) | | | |
| PART B | | | |
| Plantaren 2000N UP | Decyl Glucoside | Surfactant | 5.00 |
| | Hydroxysultaine | | |
| JARPLEX CU-WD | Cupuaçu Butter Glycerides | Conditioner | 3.00 |
| Steol CS-230 | Sodium Laureth Sulfate | Surfactant | 20.00 |
| Crodasinic LS-30 | Sodium Lauroyl Sarcosinate | Surfactant | 10.00 |
| Crosultaine C-50 | Cocamidopropyl | Surfactant | 7.00 |
| | Hydroxysultaine | | |
| PART C | | | |
| Fragrance 115620 | Fragrance | Fragrance | 0.20 |
| Color | — | Color | QS |
| Germaben II E | Propylene Glycol (and) | Preservative | 1.00 |
| | Diazolidinyl Urea (and) | | |
| | Methylparaben (and) | | |
| | Propylparaben | | |
| | | | 100 |

### Procedure:

Add the Part A water to a suitable vessel. Begin mixing and sprinkle in the Xanthan Gum. Mix until uniform and add the citric acid. Continue mixing and heat to 65-70° C. Mix until uniform. Continue mixing and stop heating. Continue mixing and add Part B in order. Mix until uniform avoiding aeration. Continue mixing and cool to 45° C. Continue mixing and add the Part C ingredients. Mix until uniform. pH as is ˜7.8 and Viscosity ˜2,550 cps Sp #3 @ 12 rpm.

### Example XIII

### Conditioning Shampoo

| INGREDIENTS | INCI Name | Function | % |
|---|---|---|---|
| PART A | | | |
| R.O. Water | Water | Solvent | 41.50 |
| Xanthan Gum | Xanthan Gum | Viscosity | 0.70 |
| | | Control | |
| Citric Acid (25% | Citric Acid | pH Control | 0.10 |
| aq soln) | | | |
| PART B | | | |
| Crosultaine C-50 | Cocamidopropyl | Surfactant | 12.50 |
| | Hydroxysultaine | | |
| JARPLEX CU-WD | Cupuaçu Butter Glycerides | Conditioner | 4.00 |
| Steol CA-30 | Sodium Laureth Sulfate | Surfactant | 30.00 |
| Stepanol CA | Sodium Lauryl Sulfate | Surfactant | 10.00 |
| PART C | | | |
| Fragrance 115620 | Fragrance | Fragrance | 0.20 |
| Color | — | Color | QS |
| Germaben II E | Propylene Glycol (and) | Preservative | 1.00 |
| | Diazolidinyl Urea (and) | | |
| | Methylparaben (and) | | |
| | Propylparaben | | |
| | | | 100 |

### Procedure:

Add the Part A water to a suitable vessel. Begin mixing and sprinkle in the Xanthan Gum. Mix until uniform and add the citric acid. Continue mixing and add the remainder of Part A. Continue mixing and heat to 65-70° C. Mix until uniform. Continue mixing and stop heating. Continue mixing and add Part B in order. Mix until uniform avoiding aeration. Continue mixing and cool to 45° C. Continue mixing and add the Part C ingredients. Mix until uniform. The pH as is ˜5.8 and Viscosity ˜4,000 cps Sp #3 @ 30 rpm.

### Example XIV

### 25% Butter Cream

| INGREDIENTS | INCI Name | Function | % |
|---|---|---|---|
| PART A | | | |
| R.O. Water | Water | Solvent | 63.91 |
| Glycerin | Glycerin | Moisturization | 3.00 |
| Pemulen TR-2 | Acrylates/C10-30 Alkyl | Viscosity | 0.20 |
| | Acrylate Crosspolymer | | |
| NaOH (50% aq. Soln) | Sodium Hydroxide | pH Control | 0.04 |
| PART B | | | |
| JARPLEX SB35 | Butyrospermum Parkii | Emollient | 25.00 |
| | (Shea Butter) | | |
| JARPLEX CU-WD | Cupuaçu Butter | Emollient | 5.00 |
| | Glycerides | | |
| DowCorning 200 | Dimethicone | Emollient | 1.00 |
| Fluid (1000 cps) | | | |
| Polyaldo 10-1-10 | Polyglyceryl-10 Oleate | Emulsifier | 0.50 |
| Mixed Tocopherols 70 | Tocopherols | Antioxidant | 0.20 |
| PART C | | | |
| Fragrance SZ18197 | Fragrance | Fragrance | 0.10 |
| Yland YIang | Fragrance | Fragrance | 0.05 |
| Color | — | Color | QS |
| Germaben II E | Propylene Glycol (and) | Preservative | 1.00 |
| | Diazolidinyl Urea (and) | | |
| | Methylparaben (and) | | |
| | Propylparaben | | |
| | | | 100 |

### Procedure:

Add the Part A water to a suitable vessel. Begin mixing and sprinkle in the Jaguar. Mix until uniform and add the citric acid. Continue mixing and add the remainder of Part A. Continue mixing and heat to 75-80° C. Mix until uniform. Continue mixing and stop heating. Continue mixing and add Part B in order. Mix until uniform avoiding aeration. Continue mixing and cool to 45° C. Continue mixing and add the Part C ingredients. Mix until uniform. pH as is ˜3.4 and Viscosity ˜5,200 cps Sp #3 @ 30 rpm.

### Example XV

### Moisturizing Lotion

| INGREDIENTS | INCI Name | Function | % |
|---|---|---|---|
| PART A | | | |
| R.O. Water | Water | Solvent | 81.85 |
| Glycerin | Glycerin | Moisturization | 2.00 |
| Ultrez 10 | Carbomer | Viscosity | 0.30 |
| Triethanolamine 99% | Triethanolamine | pH Control | 0.50 |
| PART B | | | |
| JARPLEX CU-WD | Cupuaçu Butter | Emollient | 3.00 |
| | Glycerides | | |
| Safflower Oil | Carthamus Tinctorius | Emollient | 5.00 |
| | (Safflower) Seed oil | | |
| Apricot Kernel Oil | Prunus Armeniaca | Emollient | 2.00 |
| | (Apricot) Kernel oil | | |
| Olive Oil | Olea Europaea | Emollient | 2.00 |
| | (Olive) Fruit oil | | |
| Jojoba Oil | Simmondsia Chinensis | Emollient | 1.00 |
| | (Jojoba) seed oil | | |
| Stearic Acid | Stearic Acid | Emuslsifier | 1.00 |
| Mixed Tocopherols 70 | Tocopherols | Antioxidant | 0.20 |
| PART C | | | |
| Fragrance SZ18197 | Fragrance | Fragrance | 0.10 |
| Yland YIang | Fragrance | Fragrance | 0.05 |
| Color | — | Color | QS |
| Germaben II E | Propylene Glycol (and) | Preservative | 1.00 |
| | Diazolidinyl Urea (and) | | |
| | Methylparaben (and) | | |
| | Propylparaben | | |
| | | | 100 |

### Procedure:

Add the Part A water to a suitable vessel. Begin mixing and sprinkle in the Ultrez. Mix until uniform and add the TEA. Continue mixing and add the remainder of Part A. Continue mixing and heat to 65-70° C. Mix until uniform. Continue mixing and stop heating. Combine the Part B ingredients and heat with mixing to 65-70° C. Continue mixing Part A and add Part B. Mix for at least 15 minutes at temperature. Continue mixing and cool to 45° C. Continue mixing and add the Part C ingredients. Mix until uniform. pH as is ˜7.25 and Viscosity ˜180,000 cps Sp #3 @ 0.6 rpm.

### Example XVI

Functionality is a key contributor to the efficacy of many oils. One key functional indicator is the amount of Unsaponifiables (which includes triterpene esters and other unique materials) in Shea butter or stearin or olein. The amount of Unsaponifiables was measured (AOCS Test Method Ca 6a-40 was used to measure Unsaponifiables) in the starting oil or butter as well as the corresponding mono-di-glycerin transesters to quantify that the functional material were carried over.

The results are as follows:

| | SB-35 transester | SB-15 transester | | |
|---|---|---|---|---|
| | Melting pt. 38 C. | Melting pt. 36 C. | SB-35 | SB-15 |
| Unsaponifiables | 4.9 | 6.5 | 5.4 | 7.4 |

Similar results are obtained with other butters, oils, or triglycerides due to the gentle nature of this process.

### Example XVII

JARPLEX SB-WD is mixed at 5% concentration (wt/wt) with hot water or dilute stock white water using standard mixing/agitation equipment. The resulting dispersion is sprayed onto a paper tissue web just prior to the wet line on a traditional fourdrinier paper machine. The resulting tissue would have the emollient characteristics of shea butter incorporated into the web and that would be transferred to the consumer upon use.

### Example XVIII

JARPLEX SB-WD is heated to melting and mixed at 5% concentration (wt/wt) with warm water by shaking in a small spray bottle. The resulting dispersion can be sprayed onto a textile substrate (i.e. wash cloth, inside surface of a robe). Due to it's particulate nature the Shea Butter will tend to remain on the surface of the substrate rather than penetrating. The emollient characteristics of the Shea Butter is transferred to the consumer upon use. Further, as the oil is water dispersible, removal of the material by standard home clothes washing methods is enhanced.

Although the invention herein has been described with references to particular embodiments, it is to be understood that these embodiments are merely illustrative of the principles and application of the present invention. It is therefore to be understood that numerous modifications may be made to the embodiments and that other compositions may be devised without departing from the spirit and scope of the present invention as defined by the claims.

1. A composition comprising reaction products from a reaction of a natural butter or natural oil with glycerin in the presence of a basic catalyst and wherein the reaction products retain the unsaponifiable portion of said natural butter or natural oil.

2. The composition of claim 1, wherein said reaction product is a mixture of mono- and diglycerides.

3. The composition of claim 1, wherein said basic catalyst is selected from the group consisting of sodium hydroxide and potassium hydroxide.

4. The composition of claim 3, wherein said basic catalyst is potassium hydroxide.

5. (canceled)

6. The composition of claim 1, wherein said natural oil or natural butter is selected from the group consisting of shea butter, shea stearin, shea olein, shea oil and cupuacu butter.

7. (canceled)

8. The composition of claim 6, wherein said natural oil or natural butter is cupuacu butter.

9. (canceled)

10. The composition of claim 8, wherein said composition has a maximum Color (Gardner value) not exceeding 20, a maximum acid value not exceeding 3 (mg KOH/g), a saponification value in the range of 160-180 (meq/kg), a pH in the range of 6.5-8.5 and a moisture content not exceeding 1.0%.

11. A personal care product comprising appropriate ingredients in a liquid carrier, said personal care product including an effective emulsifying amount of the reaction product of claim 10.

12-13. (canceled)

14. The personal care product of claim 11, wherein said reaction product is made from cupuacu butter.

15. (canceled)

16. The personal care product of claim 14, wherein said reaction product has a maximum Color (Gardner value) not exceeding 20, a maximum acid value not exceeding 3 (mg KOH/g), a saponification value in the range of 160-180 (meq/kg), a pH in the range of 6.5-8.5 and a moisture content not exceeding 1.0%.

17. A cosmetic product comprising cosmetically acceptable ingredients in a liquid carrier, said product including an effective emulsifying amount of the reaction product of claim 10.

18. The cosmetic product of claim 17, wherein said liquid carrier is an aqueous carrier.

19. The cosmetic product of claim 17, wherein said liquid carrier is a non-aqueous carrier.

20. The cosmetic product of claim 17, wherein said product is a shampoo.

21. The cosmetic product of claim 17, wherein said product is a shower gel.

22. The cosmetic product of claim 17, wherein said product is a topical product.

23. The cosmetic product of claim 17, wherein said product is a hair conditioner.

24. The cosmetic product of claim 17, wherein said product is a conditioning shampoo.

25. The cosmetic product of claim 17, wherein said product is a butter cream.

26. The cosmetic product of claim 17, wherein said product is a moisturizing cream.

27. A method for making the natural butter or natural oil extract of claim 10 self-emulsifiable into water while retaining the unsaponifiable portion unchanged by reacting the natural butter or natural oil extract with glycerin to convert the triglycerides present to a mixture of mono- and diglycerides.

28. A self-emulsifiable composition comprising the reaction products of claim 10.

29. (canceled)

30. A cosmetic or dermatological W/O emulsion for impregnating or moistening tissues, paper and textiles, wherein the emulsion comprises (a) a water phase, (b) at least one oil phase which comprises one or more oils, one or more lipids and combinations thereof, and (c) an emulsifier system comprising the reaction product of claim 10.
